(19) United States
(12) Plant Patent Application Publication
(10) Pub. No.: US 2016/0278263 P1
(43) Pub. Date: Sep. 22, 2016

(54) Hop plant named 'HBC 431'

(50) ;

(71) Applicants: Eugene G. Probasco, Yakima, WA (US); Jason Perrault, Toppenish, WA (US)

(72) Inventors: Eugene G. Probasco, Yakima, WA (US); Jason Perrault, Toppenish, WA (US)

(21) Appl. No.: 14/545,737

(22) Filed: Jun. 11, 2015

Related U.S. Application Data
(60) Provisional application No. 62/177,538, filed on Mar. 16, 2015.

(51) Int. Cl. A01H 5/00

(52) U.S. Cl. PLT/236

(57) ABSTRACT

A new hop plant named ‘HBC 431’ is disclosed. The cones of ‘HBC 431’ mature in mid to late September, and yields a crop of 1,600 to 2,000 pounds per acre. ‘HBC 431’ is used for its aromatic quality.

### CROSS-REFERENCE TO RELATED APPLICATIONS

None

### STATEMENT REGARDING FEDERALLY SPONSORED RESEARCH OR DEVELOPMENT

None

### GENUS AND SPECIES

Humulus lupulus

### VARIETY DENOMINATION

‘HBC 431’

### BACKGROUND OF THE INVENTION

‘HBC 431’ is a product of a controlled breeding program carried out by the inventors in the Yakima Valley of Washington State. ‘HBC 431’ was one of several seedlings resulting from a open pollination cross made in 2004 with female parent YCR 223 (unpatented). A single plant of ‘HBC 431’ was selected in 2008, and in 2009 was expanded to 30 plants, which were planted in the area of Toppenish, Wash. The plants were observed and evaluated for several years, and in 2011 were expanded for further observation and evaluation in the Topennish, Wash. area. A 1 acre test plot of ‘HBC 431’ was established in 2011. Throughout several generations of asexual propagation, ‘HBC 431’ has been observed to retain its distinctive characteristics and remain true to type.

### BRIEF DESCRIPTION OF THE PHOTOGRAPH

FIG. 1 illustrates a mature ‘HBC 431’ hop plant grown on a trellis; and

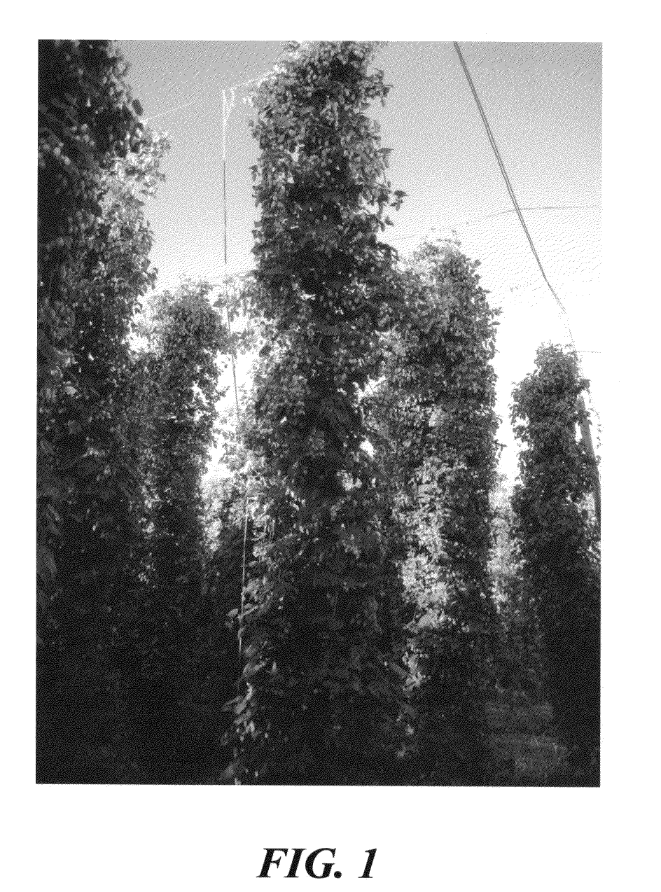

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2011-2014 growing seasons at Toppenish, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. All color references are based on The Royal Horticultural Society Color Chart.
Ploidy: Diploid
Use: Brewing
Harvest date: September 18 to 23 (during 2011 to 2014 growing seasons at Toppenish, Wash.)
Crop yield: 1600 to 2000 pounds per acre
Disease susceptibility: ‘HBC 431” is resistant to powdery mildew and normally susceptible to downy mildew.
Analytical characteristics: Alpha acid (as % of cone weight) 13.9% to 15.4% Beta acid (as % of cone weight) 4.1% to 5.1% Cohumulone (as % of alpha acids) 23.2% to 24.5%
Bine:
   Color.—144C.
   Stripe present.—Yes.
   Stripe color.—183D.
   Stipule direction.—Up-Split/Forked.
   Stipule color.—144B.
   Bine diameter.—6.75 mm at base; 6.75 mm at nine feet; 5.75 mm at terminal end of eighteen feet.
Leaf:
   Arrangement.—Opposite.
   Shape.—Palmately Lobed — Cordate.
   Average length of mature leaf.—11.9 cm.
   Average width of mature leaf.—15.6 cm.
   Color of mature leaf upper surface.—147A.
   Color of mature leaf lower surface.—147B.
   Color of immature leaf upper surface.—147A.
   Color of immature leaf lower surface.—147B.
   Number of lobes.—1-3.
   Margin.—Serrate.
   Serrations per inch.—3.64.
   Average petiole length (mature).—6.44 cm.
   Petiole color at base.—183D.
   Venation.—Palmate.
   Vein color.—14$D.
Cone:
   Avg. Length.—3.64 cm.
   Avg. diameter.—1.6 cm.
   Avg cone weight.—0.64 mg.
   Bract tip color.—144B.
   Bract base color.—142D.
   Bracteole color.—142D.
   Cone shape.—Ovoid.
   Bract shape.—Ovate.
   Bract tip shape.—Acuminate to Cuspidate.
   Bract tip position.—Loosely to moderately appressed.
   Bracteole shape.—Ovate.

What is claimed is:

1. A new and distinct hop plant as shown and described herein.
